(19) United States
(12) Patent Application Publication
(10) Pub. No.: US 2022/0143082 A1
(43) Pub. Date: May 12, 2022

(54) CELL TREATMENTS AND THERAPEUTIC REINFUSION METHODS

(71) Applicants: Todd Alamin, Woodside, CA (US); Avram Allan Edidin, Houston, TX (US); Hugues Malandain, Grans (FR); Healey Montague-Alamin, Cambridge, MA (US)

(72) Inventors: Todd Alamin, Woodside, CA (US); Avram Allan Edidin, Houston, TX (US); Hugues Malandain, Grans (FR); Healey Montague-Alamin, Cambridge, MA (US)

(21) Appl. No.: 17/580,419

(22) Filed: Jan. 20, 2022

Related U.S. Application Data
(63) Continuation of application No. PCT/US2020/042913, filed on Jul. 21, 2020.
(60) Provisional application No. 62/877,011, filed on Jul. 22, 2019.

(51) Int. Cl. A61K 35/13; C12M 1/00; C12N 5/09

(52) U.S. Cl. CPC A61K 35/13; C12N 5/0693; C12M 45/20

(57) ABSTRACT

Tumor tissue, including soft and/or bony tissue, are harvested from a subject and morcellated. The morcellated tissue is placed in a cartridge which is placed in a containment chamber of a tumor tissue processing device. Cancer cells in the morcellated tumor tissue are destroyed without destroying tumor antigens therein. These cells are destroyed cryogenically by exposing the cartridge to a cooling fluid such as liquid nitrogen, optionally with a warming cycle, and optionally with more than one freezing/thawing cycle. The treated tissue and/or cells are then extracted from the cartridge and reintroduced to the subject after they have reached a threshold condition. The treated tissue and/or cells can be reintroduced via a containment sleeve or a reimplantation bag.

### CROSS-REFERENCE

This application is a continuation of PCT Application No. PCT/US2020/042913, filed Jul. 21, 2020; which claims priority to U.S. Provisional Application No. 62/877,011 filed Jul. 22, 2019; which are fully incorporated herein by reference.

### BACKGROUND

Cancer is a leading cause of death in the United States, other countries, and the world. Traditionally, cancer has been treated with surgery, chemotherapy, radiation therapy, or combinations thereof. Recently, immunotherapy-based approaches have gained success and traction. These include monoclonal antibodies, non-specific immunotherapies, oncolytic virus therapies, T-cell therapies such as chimeric antigen receptor (CAR) T-cell therapies, and tumor antigen vaccines. The effectiveness of these therapies is in large part due to the personalization of these therapies to the target patient. Despite the notable progress, a “cure” for cancer has yet to be realized and improvements to cancer therapies are still desired.

Relevant publications include: US20190023670, US20180362519, US20180044630, US20140227781, US20130122049, and U.S. Pat. No. 6,036,681.

### SUMMARY

The present disclosure provides methods, systems, and devices for treating tissue and/or cells extracted from a subject for subsequent therapeutic infusion back to the subject, often as a tumor antigen vaccine to treat cancer.

Aspects of the present disclosure provide methods for treating tissue or cells for therapeutic reinfusion. Tumor tissue, including soft and/or bony tissue, may be harvested from a subject. The tumor tissue may be morcellated, generally in a containment chamber or a specific morcellation device. Cancer cells in the morcellated tumor tissue may be destroyed or otherwise devitalized without destroying tumor antigens therein. The cancer cells may be destroyed by cooling (e.g., cryogenically) such as by liquid nitrogen. This devitalization or destruction of the cells may be potentiated by one or more freeze/thaw cycles, and these cycles may be further optimized by adjusting the rate of cooling and warming in the freeze/thaw cycles and/or the length, number, and/or pattern of the freeze/thaw cycles. The freeze/thaw cycles may be implemented with the containment chamber which houses the morcellated tissue. The tumor tissue with devitalized or destroyed cancer cells and preserved antigens may then be extracted from the containment chamber and reintroduced to the subject. This tissue may be extracted only after it has reached a threshold condition, such as a threshold temperature or a submersion level in the processing chamber, and/or a number of freeze/thaw cycles of a specified length and/or pattern. In some cases, this tissue is reintroduced with a containment element such as a sleeve, which may be optimized to intensify the immune response to the re-implanted tissue.

Aspects of the present disclosure also provide systems and devices for treating tissue or cells for therapeutic reinfusion. An exemplary system may comprise one or more of: a cartridge for storing and processing harvested tumor tissue; a tissue morcellator within the cartridge, operatively couplable to the cartridge, or operated independently of the cartridge; a housing including a containment chamber to removably hold the cartridge and optionally a driver for the tissue morcellator; a cooling fluid port fluidically coupled to the containment chamber for introduction of a cooling agent into the containment chamber to cool the cartridge; and/or a user interface for one or more of (i) displaying one or more of time, a temperature in the containment chamber, and/or morcellation status, and/or (ii) controlling one or more of a timer, the temperature, and/or the driver for the tissue morcellator, and/or (iii) monitoring and driving the characteristics of one or more freeze/thaw cycles. These characteristics may include the rate and duration of freezing, the rate and duration of thawing, target temperatures for freezing and thawing, etc. The system may further comprise a warm air or fluid port fluidically coupled to the containment chamber for introduction of a warm air or fluid to the containment chamber to warm or thaw the cartridge. Alternatively or in combination, a warming element, such as an inductive and/or resistive heating element, may be provided to warm or thaw the cartridge. The cartridge may be in the form of a tube, tray, and/or mesh cylinder.

Aspects of the present disclosure provide methods for treating cancer. An exemplary method may comprise a step of reintroducing into a subject tumor tissue harvested from the subject, the reintroduced tumor tissue having had cancer cells thereof devitalized without destroying tumor antigens in said cancer cells.

The tumor tissue harvested from the subject may comprise one or more of soft tissue or bony tissue. The method may further comprise a step of harvesting the tumor tissue from the subject. The method may further comprise a step of morcellating the tumor tissue harvested from the subject prior to reintroducing the tumor tissue into the subject. The tumor tissue may be morcellated by placing the harvested tumor tissue in a containment chamber, where the tumor tissue is morcellated.

The method may further comprise a step of devitalizing cancer cells in the tumor tissue harvested from the subject. Devitalizing the cancer cells in the tumor tissue may comprise destroying the cancer cells. Devitalizing cancer cells in the tumor tissue may comprise cooling the tumor tissue. The tumor tissue may be cooled after being morcellated. The cooled tumor tissue may be warmed. The method may further comprise a step of repeating one or more cool and warm cycles to the tumor tissue. The one or more cool and warm cycles may conform to a specified range of speeds of cooling and warming, optimized to devitalize or kill cells without destroying tumor antigens. The tumor tissue with the cancer cells devitalized may be reintroduced into the subject after said tumor tissue has reached a threshold number of cool and warm cycles and/or after said tumor tissue has reached a threshold temperature and optionally for a predetermined period of time. The tumor tissue may be cooled by cryogenically treating the tumor tissue, such as with liquid nitrogen. The tumor tissue with the cancer cells thereof devitalized may be reintroduced with a containment element, such as a containment sleeve.

Aspects of the present disclosure provide systems for treating tissue or cells for therapeutic reinfusion. An exemplary system may comprise: a cartridge for storing and processing harvested tumor tissue; a housing including a containment chamber to removably hold the cartridge;

a cooling fluid port fluidically coupled to the containment chamber for introduction of a cooling agent into the containment chamber to cool the cartridge; and, a user interface. The user interface may be configured for one or more of: (i) displaying one or more of time, a temperature in the containment chamber, or the phase of the freezing/thawing cycle, or (ii) controlling one or more of a timer or the temperature in the containment chamber, or (iii) monitoring and driving characteristics of one or more freeze/thaw cycles.

The system may further comprise a tissue morcellator within the cartridge. The housing may include a driver for the tissue morcellator. The user interface may be configured to display a morcellation status of the harvested tumor tissue. The user interface may be configured to control the driver for the tissue morcellator.

The system may further comprise a warm air port fluidically coupled to the containment chamber for introduction of a warm air to the containment chamber to warm or thaw the cartridge.

The cartridge may be in various forms, such a tube, a tray, a mesh cylinder or the like.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings of which:

FIG. 1A is a schematic of a tumor tissue processing device, according to embodiments of the present disclosure.

FIG. 1B is a top view of the containment chamber of the tumor tissue processing device of FIG. 1A.

FIG. 2 is a side view of an exemplary containment chamber for a tumor tissue processing device, according to embodiments of the present disclosure.

FIG. 3 is a flow chart of a tumor tissue processing and reinfusion method, according to embodiments of the present disclosure.

FIG. 4A shows a perspective view of a tissue processing system, according to embodiments of the present disclosure.

FIG. 4B shows an exploded view of the tissue processing system of FIG. 4A.

FIGS. 4C, 4D, and 4E show a perspective, (left) side, and (right) side views, respectively, of the console shell of the tissue processing system of FIG. 4A.

FIGS. 4F and 4G show perspective views of the multi-purpose lid of the tissue processing system of FIG. 4A.

FIG. 4H shows a perspective view of the lid lock of the tissue processing system of FIG. 4A.

FIGS. 41, 4J, and 4K show perspective views of the tissue processing system of FIG. 4A in a cryo/cooling cycle position, a transition position, and a warming cycle position, respectively.

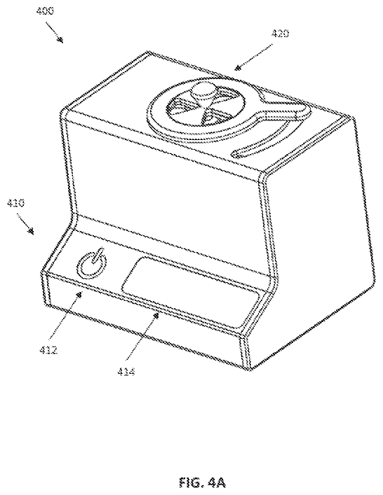

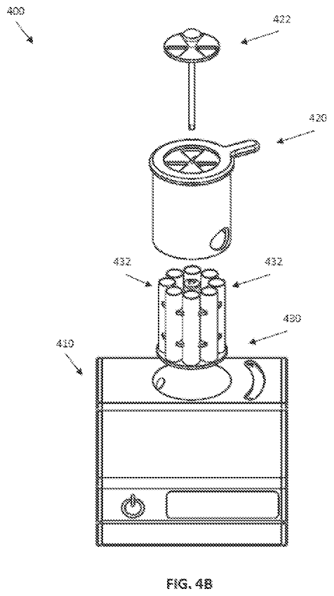

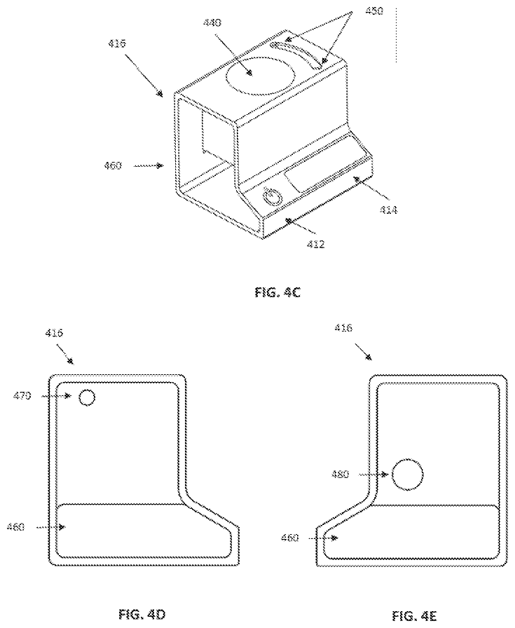

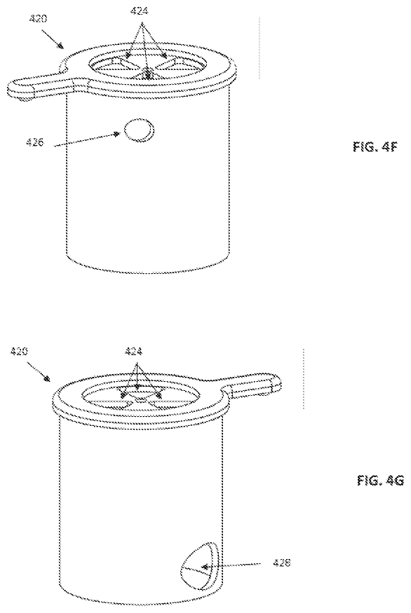

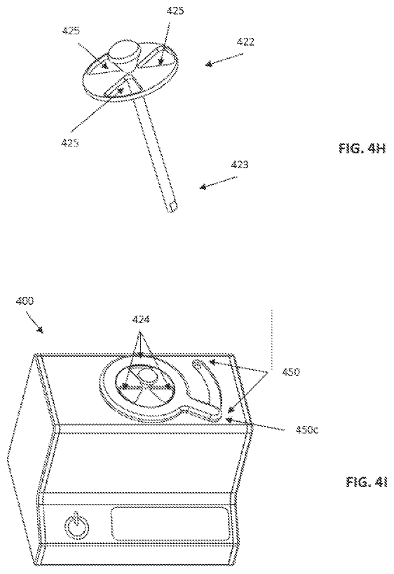

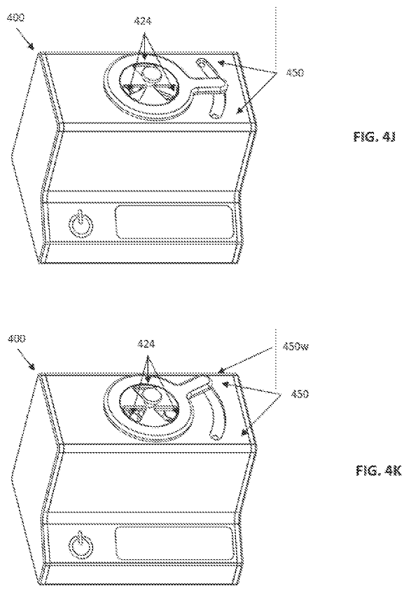

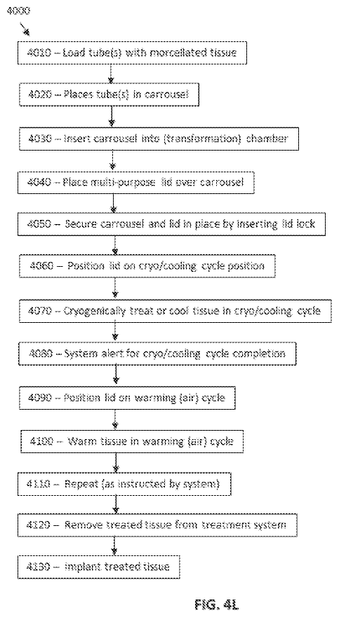

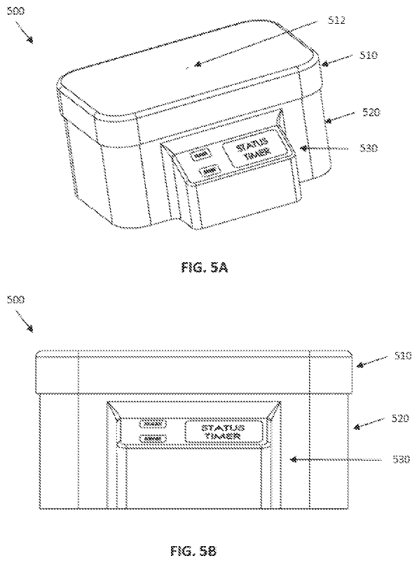

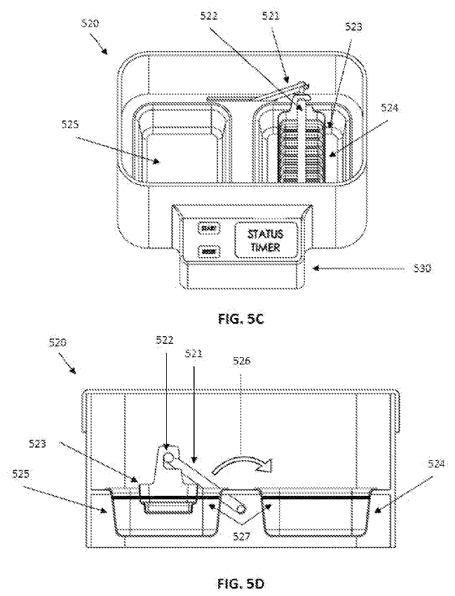

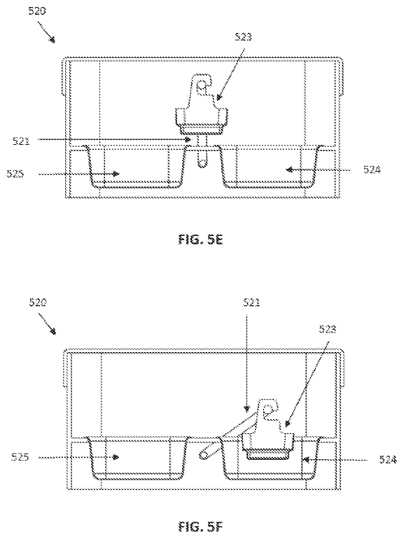

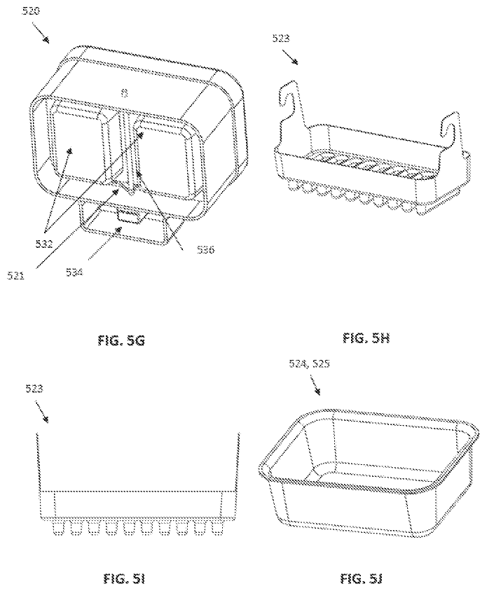

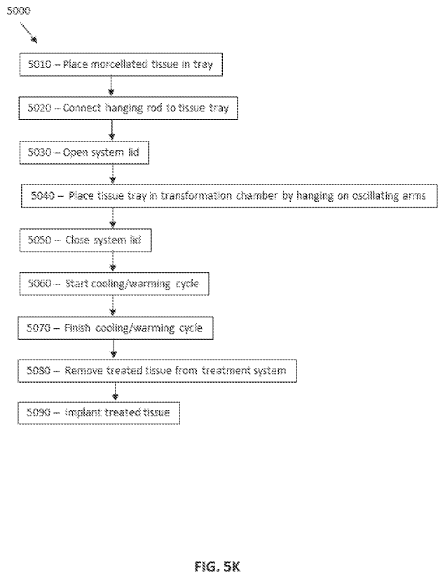

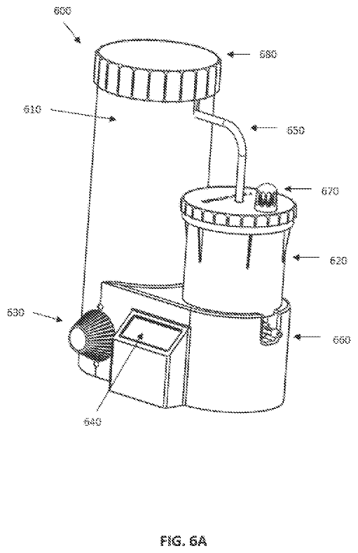

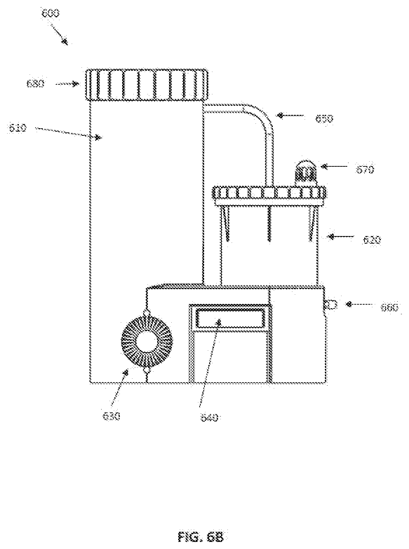

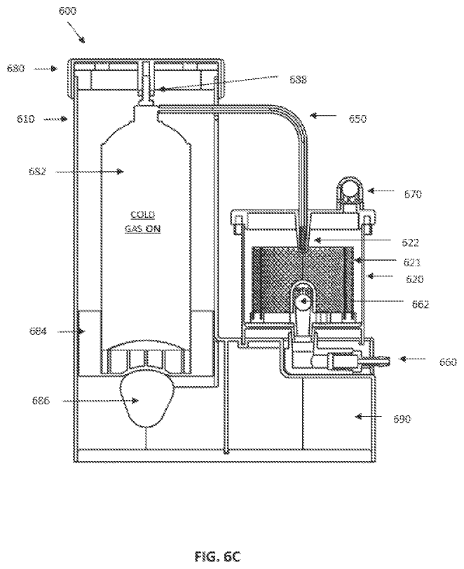

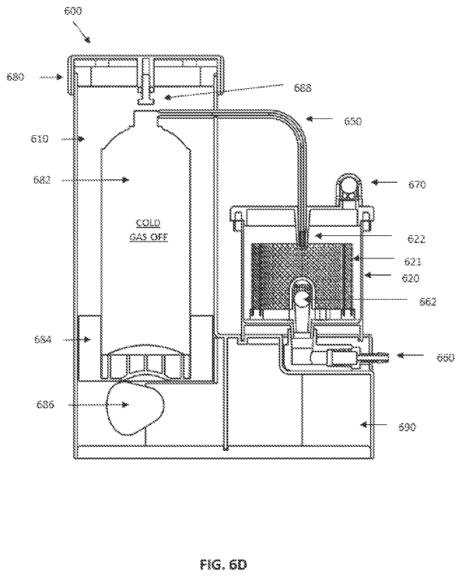

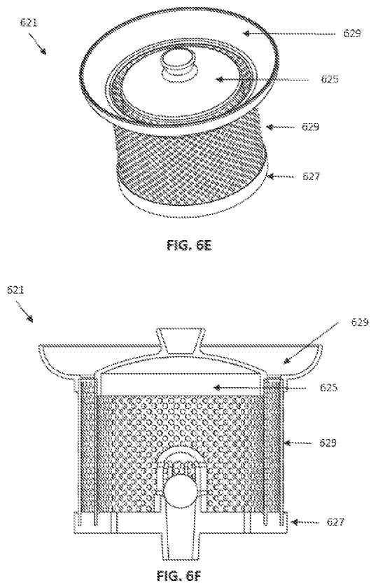

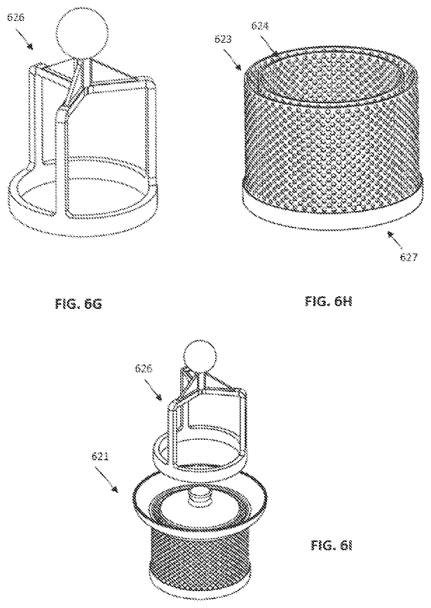

FIG. 4L shows a flow chart of an exemplary method of use of the tissue processing system of FIG. 4A, according to embodiments of the present disclosure.

FIG. 5A shows a perspective view of a tissue processing system, according to further embodiments of the present disclosure.

FIG. 5B shows a front view of the tissue processing system of FIG. 5A.

FIG. 5C shows a top view of the transformation chamber of the tissue processing system of FIG. 5A.

FIGS. 5D, 5E, and 5F show side section views of the transformation chamber of the tissue processing system of FIG. 5A in a cryo/cooling cycle position, a transition position, and a warming cycle position, respectively.

FIG. 5G shows a bottom, perspective view of the uncovered transformation chamber of the tissue processing system of FIG. 5A.

FIGS. 5H and 5I show perspective and side views, respectively, of a tissue tray for the transformation chamber of the tissue processing system of FIG. 5A.

FIG. 5J shows a perspective view of a cover for the transformation chamber of the tissue processing system of FIG. 5A.

FIG. 5K shows a flow chart of an exemplary method of use of the tissue processing system of FIG. 5A, according to embodiments of the present disclosure.

FIGS. 6A and 6B show perspective and side views, respectively, of a tissue processing system, according to further embodiments of the present disclosure.

FIGS. 6C and 6D show side section views of the tissue processing system of FIGS. 6A and 6B in “cold gas on” and “cold gas off” positions, respectively.

FIGS. 6E and 6F show perspective and side views, respectively, of a mesh assembly for holding tissue to be processed for the tissue processing system of FIGS. 6A and 6B, according to embodiments of the present disclosure.

FIG. 6G shows a perspective view of a ring tissue/bone packer suitable for use with the mesh assembly of FIGS. 6E and 6F, according to embodiments of the present disclosure.

FIG. 6H shows a perspective view of the mesh assembly of FIGS. 6E and 6F uncovered.

FIG. 6I shows a perspective view of the ring tissue/bone packer of FIG. 6G in use with the mesh assembly of FIGS. 6A and 6B.

FIG. 6J shows a flow chart of an exemplary method of use of the tissue processing system of FIGS. 6A and 6B, according to embodiments of the present disclosure.

### DETAILED DESCRIPTION

The present disclosure provides methods, systems, and devices for treating tissue and/or cells extracted from a subject for subsequent therapeutic infusion back to the subject, often as a tumor antigen vaccine to treat cancer. In particular, provided are devices configured to kill tumor cells while preserving tumor antigens to allow for reimplantation (also referred to herein as “reinfusion” or “reintroduction”) of the killed tumor cells to the patient to stimulate an immune response to the tumor.

Suitable tumor tissue processing devices should meet many device requirements. Devices should predictably prepare tissue for the steps of the tissue processing method to kill the tumor cells, which will often include cryo treatment, optionally involving one or more freeze/thaw cycles. Devices should facilitate cryo treatment of the tumor tissue in a predicable way to allow for complete freezing of tissue followed by cell lysis, resulting in the destruction of the viability of cells. Devices should be able to deliver the resultant tissue for re-implantation or reinfusion. Devices may have implantation cuff(s) for containment of tumor tissue containment and ease of reimplantation or reinfusion.

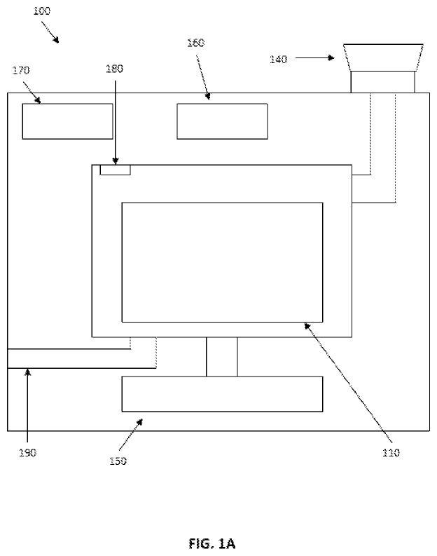

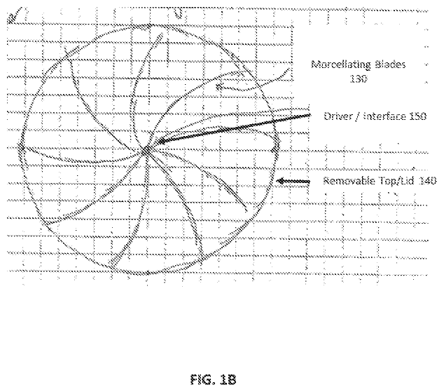

Many key elements of the tumor tissue processing device are shown in FIG. 1A, which shows an exemplary tumor tissue processing device 100. A key component is a containment chamber 110 which can interface with a replaceable cartridge for tissue morcellation, tissue storage, and/or other processing. Harvested tumor tissue may be placed in the cartridge within which the stages of tumor tissue treatment may be performed. The tumor tissue as such can stay within the cartridge for the entire treatment so that complete treatment is assured without contamination of untreated tissue. Typically, tissue never leaves cartridge until treatment is finished, though in some instances, the tissue may be morcellated with an independent device and placed in the cartridge for treatment such as with cooling and warming cycle(s). Two types of cartridges may be provided—one for bone and hard tissues, one for soft tissue.

In some embodiments, the tissue is morcellated in the same cartridge as for the downstream (cooling and warming) treatment. The bone/hard tissue cartridge may have mechanical blade(s) 130 that spins and breaks up tissue into smaller pieces that can predictably be treated by liquid nitrogen or other cryogenic/cooling fluid as shown in FIG. 1B. The size(s) of the pieces can be determined by lab studies looking at depth of cryo treatment penetration (typically with liquid nitrogen as introduced by the funnel 140 as shown in FIG. 1A) time course to assure complete treatment. The blade(s) 130 can interact with a drill 150 in the upper part of the device 100 that spins the blade(s) 130 for the required amount of time.

The soft tissue cartridge can have a series of sharp sieves that cut tissue into the required sizes based again upon temperature penetration studies, but also for handling requirements—small pieces easier to handle. These sharp sieves can be activated by mechanical lever(s) 150 that are depressed by the clinician or automated into the function of the device.

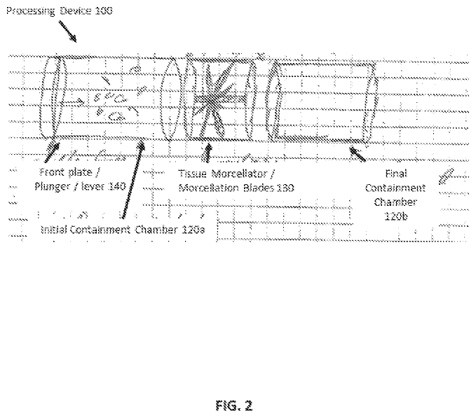

The tumor tissue processing device may have a first part to allow for the initial device function to work—allowing for the cartridge is placed into the device, and then performing the tissue morcellation function with the assistance of the cartridge (for example, the initial containment chamber 120a as shown in FIG. 2). The tumor tissue processing device may include a lever or plunger 150 (in the form of a top/front plate, for example) which can be deployed to move the cartridge into the second part of the device 100, which immerses the cartridge in liquid nitrogen (or some other cooling agent) which then is completely submerged for the required time (for example, the final containment chamber 120b as shown in FIG. 2). As shown in FIG. 1A, there may be a timer 160 on the device 100 which produces a visual and/or audible signal when the treatment is done. As shown in FIG. 1A, the device 100 may also have a temperature readout 170 (as measured by sensor(s) 180 which may measure temperature as well as other conditions such as pH, oxygenation, water content, etc.), as well as one that reads out the number of freezing/heating cycles. The cooling agent can then be drained from the tissue containment chamber of the device (suction/gravity port 190 for drainage as shown in FIG. 1B) to minimize the likelihood of burning the clinician, and the cartridge can then delivered be from the device to allow for tissue reimplantation or reinfusion. Optionally, the apparatus may facilitate one or more freeze/thaw cycles of specified characteristics. Optionally, the tumor tissue can then be delivered into an implantable bag to facilitate reimplantation. The reimplantation bag can be made of either resorbable material or nonabsorbable material to allow for easy explantation. This material may be optimized to heighten or potentiate the immune response to the replanted tissue. The bag may be porous to allow for easy bioavailabilty, which is often critical for stimulating immune response. The bag or sleeve may fit over the top of the containment chamber after the top of the chamber has been removed or opened. The device may further include a cartridge temperature monitor to confirm appropriate temperature cycling is reached to kill tumor cells, but also to allow clinician to know when temperature has returned to room temperature to allow for safe handling and reimplantation.

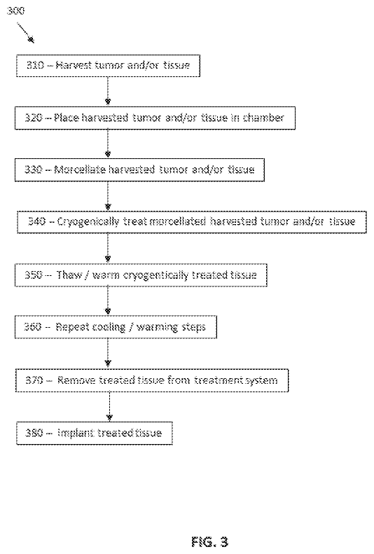

FIG. 3 shows a flow chart of a tumor tissue processing and reinfusion method 300. Tumor tissue may first be harvested, for instance, in a step 310. The tumor tissue may be bony tissue, soft tissue, or both. The harvested tumor tissue may then be placed in a containment chamber, typically within a cartridge, for instance, in a step 320. The tumor tissue may then be morcellated, for instance, in a step 330. The tumor tissue may then be treated with a cryogenic agent, such as liquid nitrogen, to kill or otherwise devitalize the cells without destroying their tumor antigens, for instance, in a step 340. The tumor tissue may be cooled to a temperature of −50° C. or below to kill, destroy, or otherwise devitalize cancerous cells in the morcellated tissue. The temperature and/or submersion level of the treated tissue can be monitored, and once reached a threshold temperature, such as −50° C. or below for at least a given period of time, such as one minute, the treated tissue may be thawed (i.e., heated or warmed) in a step 350. Once a threshold temperature is reached again, such as −50° C. or below for at least a given period of time, such as one minute, the treated tissue may be again cooled. After one or more of freezing/heating cycles or repeats of steps 340 and 350, in a step 360, the treated tissue may be collected. In some embodiments, repeated cooling and heating may not be necessary. In a step 370, the treated tissue can be removed from the cartridge and tissue treatment system The treated tissue can then be re-implanted, for instance, in a step 380. The treated tissue can be re-implanted typically within a tissue containment sleeve or bag, but also in isolation. One use may be for bone grafting in a fusion procedure.

Although the above steps describe a particular method of harvesting, processing, and reintroducing tissue in accordance with many embodiments, a person of ordinary skill in the art will recognize many variations based on the teaching described herein. The steps may be completed in a different order. Steps may be added or omitted. Some of the steps may comprise sub-steps. Many of the steps may be repeated as often as beneficial to the treatment.

Referring to FIGS. 4A-4M, a tissue processing system 400 and an exemplary method of use 4000 for the system 400 are described. The tissue processing system 400 may comprise a transformation chamber console 410, a lid 420, and a carrousel 430 for tubes 432 to hold morcellated tissue, as shown in FIG. 4B. Alternatively or in combination with using the carrousel 430 with tubes 432 to hold morcellated tissue, a mesh assembly as described herein may be used, for example, the mesh assembly described further below and with FIGS. 6A-6J. The chamber console 410 may include a power button 412 and a user interface 414 which may include a graphic display, as shown in FIG. 4A. The system 400 may further include a lid cover 422 operatively couplable to the lid 420. The chamber console 410 may comprise a console shell 416 and define a transformation chamber 440 and (cool/warm) cycle switches 450. As shown in FIGS. 4C to 4E, the console shell 416 may define a void 460. The void 460 may accommodate a cryo or cooling gas delivery system on one side and a warm air delivery system on the opposite side, as well as the electronic control components of the system 400. The system 400 may further comprise a cryo or cooling gas inlet 470 and a thawing and/or warm air inlet 480.

The lid 420 be a multi-purpose lid and have multiple purposes including closing the transformation chamber 440, switch the system 400 between a cryo/cooling cycle and a warming/thawing cycle, allowing either cryo/cooling gas or warm air to enter the transformation chamber, and/or provide venting. As shown in FIGS. 4F and 4G, the lid 420 may comprise vents 424, a cryo or cooling gas inlet 426 that can be aligned with the cryo gas inlet 470, and a warm gas inlet 428 that can be aligned with the warm gas inlet 480. As shown in FIG. 4H, the lid lock 422 may comprise a keyed shaft 423 and openings 425 which can be aligned with the vents 424 of the lid 420 in an (vents) open position. The lid 420 may be rotated relative to the (relatively stationary) lid lock 422 to open and close the vents 424. The keyed shaft 423 can orient the windows or openings 425 in order to allow predictable opening and closing of the lids of 424 as it is actuated. The lid lock 422 may be held in place, for example, by a magnet such as a rare earth element magnet at the bottom of the transformation chamber 440.

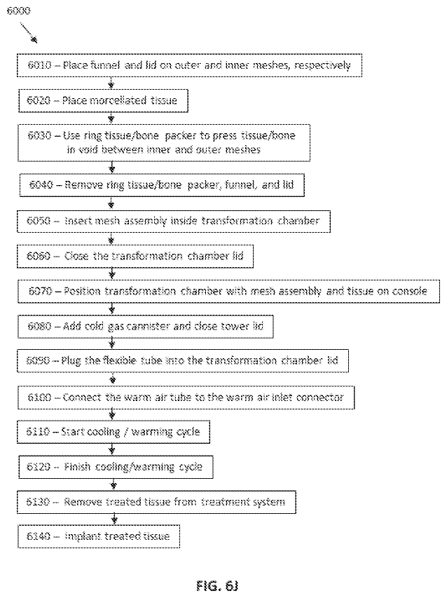

As shown in FIGS. 41 to 4K, the lid 420 may be rotated to open and close the vents 424, 425 as well as switch the system 400 between cooling and warming/thawing functions by interfacing with the cycle switches 450. The lid 420 may be positioned in the cryo or cooling cycle position 450c with the vents 424 being closed as in FIG. 4AI. While the vents 424 are closed, the vents 424 in some embodiments are not fully sealed so that there is low to no pressure buildup. In the cryo or cooling cycle position 450c, the cryo gas inlet 426 of the lid 420 may be aligned with the cryo gas inlet 470 to allow cryo or cooling gas to enter the transformation chamber 440 as in FIG. 4F, while mis-aligning the warm gas inlet 428 with the warm gas inlet 480 to prevent warm gas from entering the transformation chamber 440.

The lid 420 may be positioned in a transitional position with the lids with the vents 424 being partially opened as in FIG. 4J. In this transitional position, the cryo gas inlet 426 of the lid 420 may be mis-aligned with the cryo gas inlet 470 to prevent cryo or cooling gas from entering the transformation chamber 440 and the warm gas inlet 428 may be mis-aligned with the warm gas inlet 480 to prevent warm gas from entering the transformation chamber 440.

The lid 420 may be positioned in a warming or thawing cycle position 450w with the vents 424 being (fully) opened. A high flow of warm air may be pushed into the transformation chamber 440 and the open vents 424 can prevent a pressure buildup and also allow for thermal exchange. In this warming or thawing cycle position, the cryo gas inlet 426 of the lid 420 may be mis-aligned with the cryo gas inlet 470 to prevent cryo or cooling gas from entering the transformation chamber 440, while the warm gas inlet 428 is aligned with the warm gas inlet 480 to allow warm gas to enter the transformation chamber 440 as in FIG. 4G. The warm air may comprise hot air that is pushed into the transformation chamber 440. The warm air may be sterile and be sourced from a cannister, for instance. The warm air may comprise warmed nitrogen gas, for example. In some embodiments, a warming element such as an induction coil may be provided near the warm gas inlet 428 and/or the warm gas inlet 480 to independently warm incoming air. In some embodiments, a UV light emitter may be provided to sterilize the incoming warm air.

Referring to FIG. 4L, a method 4000 for processing tissue, such as with the system 400, is now described. In a step 410, one or more of the tubes 432 may be loaded with morcellated tissue. The morcellated tissue may be provided from any tissue morcellation device and loaded into the one or more tubes 432 in morcellated form. As noted above, alternatively or in combination with using the carrousel 430 with tubes 432 to hold morcellated tissue, a mesh assembly as described herein may be used, for example, the mesh assembly described further below and with FIGS. 6A-6J. In a step 4020, the loaded tube(s) 432 may be placed in the carrousel 430. In a step 4030, the carrousel 430 may be placed into the transformation chamber 440. In a step 4040, the multi-purpose lid 420 may be placed over the carrousel 430 in the transformation chamber 440. In a step 4050, the carrousel 430 and the lid 420 may be secured by inserting the lid lock 422. In a step 4060, the lid 420 may be positioned in the cryo or cooling cycle position 450c. In a step 4070, the morcellated tissue may be cryogenically treated or cooled in the cryo or cooling cycle so as to kill, destroy, or otherwise devitalize cancerous cells in the morcellated tissue. The tissue may be cooled to a threshold temperature as described further above with respect to method 300. In a step 4080, the system 400 may provide an alert that the cryo or cooling cycle is complete. In a step 4090, the lid 420 may be positioned in the thawing or warming cycle 450w. In a step 4100, the tissue may be thawed and/or warmed in the thawing or warming cycle. The tissue may be warmed to a threshold temperature as described further above with respect to method 300. While the use of warm air is described, the tissue may be warmed in other ways as well, such as with a heating element, for example, an inductive or resistive heating element. The system 400 may provide an alert that the thawing or warming cycle is complete, and in a step 4110, the cooling/warming (i.e., freezing/thawing) may be repeated as instructed by the system 400. In some embodiments, repeated cooling and heating may not be necessary. In a step 4120, the treated tissue can be removed from the tubes 432 and tissue treatment system 400. The treated tissue can then be re-implanted, for instance, in a step 4130. The treated tissue can be re-implanted typically within a tissue containment sleeve or bag, but also in isolation. One use may be for bone grafting in a fusion procedure.

Although the above steps describe a particular method of processing tissue in accordance with many embodiments, a person of ordinary skill in the art will recognize many variations based on the teaching described herein. The steps may be completed in a different order. Steps may be added or omitted. Some of the steps may comprise sub-steps. Many of the steps may be repeated as often as beneficial.

Referring to FIGS. 5A-5K, a tissue processing system 500 and an exemplary method of use 5000 for the system 500 are described. As shown in FIGS. 5A and 5B, the system 500 may comprise a lid 510 with a lid vent 512, a transformation chamber 520, and a control unit 530 which may include a user interface and/or a display. As shown in FIGS. 5C to 5F, the transformation chamber 520 may comprise an oscillating arm 521 coupled to a hanging rod 522 for carrying and moving a tissue tray 523 for holding morcellated tissue (shown in more detail in FIGS. 5H and 5I) between a warming pan 524 and a cooling pan 525, such as indicated by arrow 526 in FIG. 5D. FIG. 5D also shows a liquid level 527 for the warming pan 524 and the cooling pan 525 which the tissue tray 523 is not placed under so as to prevent contamination of the held tissue by the warming and cooling liquids. The cooling liquid may be liquid nitrogen, for example. FIG. 5D shows the tissue tray 523 placed in the cooling pan 525, FIG. 5E shows the tissue tray 523 in process of being moved from the cooling pan 525 and in a neutral position, and FIG. 5F shows the tissue tray 523 placed in the warming pan 524. As shown in FIG. 5G, the body of the transformation chamber 520 may comprise a pair of insulated steel pans 523 for the warming and cooling pans 524, 525 shown, for example, in FIG. 5J. The transformation chamber 520 may also include a step motor and electronic controls 534 and an actuation shaft 536 coupled to the step motor and the oscillating arm 521 to control and move the oscillating arm 521 according to the current phase of the cooling/warming cycle. As shown in FIGS. 5H and 5I, the bottom surface of the tissue tray 523 may be corrugated to provide a large surface area and optimize energy transfer during cooling and warming.

Referring to FIG. 5K, a method 5000 for processing tissue, such as with the system 500, is now described. In a step 5010, morcellated tissue may be placed in the tissue tray 523. The morcellated tissue may be provided from any tissue morcellation device and loaded into the tissue tray 523 in morcellated form. The morcellated tissue may be placed in the tray 523 such that all cavities in the corrugated bottom surface are filled and flush or sub-flush with the surface. In a step 5020, the hanging rod 522 may be coupled to the tissue tray 523. In a step 5030, the system lid 510 may be opened. In a step 5040, the tissue tray 523 may be placed in the transformation chamber 520 by hanging on the oscillating arm 520, typically in the neutral position. In a step 5050, the cooling/warming cycle may be started by the system 500. The tissue may be cooled and warmed to threshold temperatures as described further above with respect to method 300. The cooling/freezing of the tissue can kill, destroy, or otherwise devitalize cancerous cells in the morcellated tissue. In the cooling/warming cycle, the arm 521 may oscillate from the cryo/cooling pan to the thawing/warming pan in predetermined intervals to ensure that the tissue freezes and thaws. The cycle may occur once or multiple times. While the use of warm liquid in a tray is described, the tissue may be warmed in other ways as well, such as with a heating element, for example, an inductive or resistive heating element. In a step 5070, the cooling/warming cycle may be finished by the system 500. In a step 5080, the treated tissue can be removed from the tissue tray 523 and tissue treatment system 500. The treated tissue can then be re-implanted, for instance, in a step 5090. The treated tissue can be re-implanted typically within a tissue containment sleeve or bag, but also in isolation. One use may be for bone grafting in a fusion procedure.

Although the above steps describe a particular method of processing tissue in accordance with many embodiments, a person of ordinary skill in the art will recognize many variations based on the teaching described herein. The steps may be completed in a different order. Steps may be added or omitted. Some of the steps may comprise sub-steps. Many of the steps may be repeated as often as beneficial.

Referring to FIGS. 6A-6J, a tissue processing system 600 and an exemplary method of use 6000 for the system 600 are described. As shown in FIGS. 6A and 6B, the system 600 may comprise a system housing 610, a transformation chamber 620, an actuation button or control 630, a display or screen 640, a flexible tubing 650 for cold gas, a warm air inlet connector 660, a vent 670, and a cold tower lid 680. As shown in FIGS. 6C and 6D, the cold tower lid 680 may be opened and a cold gas cannister 682 may be placed within the cold tower of the system 600. The cold gas cannister 682 may hold a cooling gas such as nitrogen gas, for example. The system 600 may comprise a piston 684 couplable to the cold gas cannister 682 and operable with a cam 686. In an “on” position, the cam 686 may be actuated to advance the piston 684 and the cold gas cannister 682 to the adjustable hard stop 688, opening the top valve of the cold gas cannister and allowing cold gas to enter the transformation chamber 620 via the flexible tubing 650, as shown in FIG. 6C. The transformation chamber 620 may removably house a mesh cylinder or mesh assembly 621 for containing morcellated tissue. A cold air diffuser 622 may be provided within the transformation chamber 620 to diffuse the cold air from the cold gas canister 682. In an “off” position, the cam 686 may be actuated to retract the piston 684 and the cold gas cannister 682, allowing the top valve of the cold gas cannister to close and preventing cold gas from entering the transformation chamber 620 via the flexible tubing 650, as shown in FIG. 6D. In this “off” position, warm air may be allowed into the transformation chamber 620 via the warm air inlet (ball) valve 662 and the warm air inlet connector 660. The warm air may comprise hot air that is pushed into the transformation chamber 620. The warm air may be sterile and be sourced from a cannister, for instance. The warm air may comprise warmed nitrogen gas, for example. In some embodiments, a warming element such as an induction coil may be provided near the warm air inlet valve 662 and/or the warm air inlet connector 660 to independently warm incoming air. In some embodiments, a UV light emitter may be provided to sterilize the incoming warm air.

FIGS. 6E to 6I show the mesh cylinder or mesh assembly 621 and/or a ring (bone) tissue packer 626 to be used with the mesh assembly 621. The mesh assembly 621 may comprise an outer mesh layer 623, an inner mesh layer 624, and a base 627. The ring tissue packer 626 may be used to position and pack morcellated tissue in the space between the outer mesh layer 623 and the inner mesh layer 624. A removable funnel 625 and a lid 629 may be placed on the mesh assembly 621 to guide the tissue packing. After the morcellated tissue has been packed into said space, the mesh assembly 621 may be placed in the transformation chamber 620. The funnel 629 and the lid 625 may be removed before placement of the mesh assembly 621 into the transformation chamber 620.

Referring to FIG. 6J, a method 6000 for processing tissue, such as with the system 600, is now described. In a step 6010, the removable funnel 629 and lid 625 may be placed on the inner and outer meshes of the mesh assembly 621. In a step 6020, morcellated tissue may be placed on the removable funnel 629 and lid 625. The morcellated tissue may be provided from any tissue morcellation device and placed on the removable funnel and lid 629 in morcellated form. In a step 6030, the ring packer 626 may be used to press the morcellated (bone) tissue into the void or space between the inner and outer meshes. In a step 6040, the ring packer 626 and the funnel 629 and lid 625 may be removed from the mesh assembly 621. In a step 6050, the mesh assembly 621 may be inserted inside the transformation chamber 620. In a step 6060, the lid of the transformation chamber 620 may be closed. In a step 6070, the transformation chamber 620 with the mesh assembly 621 and tissue may be placed within the console or system housing 610. In a step 6080, the cold gas cannister 682 may be placed within the console or system housing 610 and the tower lid 680 may be closed. In a step 6090, the flexible tube 650 may be plugged into the transformation chamber lid. In a step 6100, a warm air tube may be connected to the warm air inlet connector 660. In a step 6110, the cooling/warming cycle may be started by the system 600. The tissue may be cooled and warmed to threshold temperatures as described further above with respect to method 300. The cooling/freezing of the tissue can kill, destroy, or otherwise devitalize cancerous cells in the morcellated tissue. The cycle may occur once or multiple times. While the use of warm air is described, the tissue may be warmed in other ways as well, such as with a heating element, for example, an inductive or resistive heating element. In a step 6120, the cooling/warming cycle may be finished by the system 600. In a step 6130, the treated tissue can be removed from the mesh assembly 621 and tissue treatment system 600. The treated tissue can then be re-implanted, for instance, in a step 6140. The treated tissue can be re-implanted typically within a tissue containment sleeve or bag, but also in isolation. One use may be for bone grafting in a fusion procedure.

Although the above steps describe a particular method of processing tissue in accordance with many embodiments, a person of ordinary skill in the art will recognize many variations based on the teaching described herein. The steps may be completed in a different order. Steps may be added or omitted. Some of the steps may comprise sub-steps. Many of the steps may be repeated as often as beneficial.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for treating cancer, the method comprising:
reintroducing into a subject tumor tissue harvested from the subject, the reintroduced tumor tissue having had cancer cells thereof devitalized without destroying tumor antigens in said cancer cells.

2. The method of claim 1, wherein the tumor tissue harvested from the subject comprises one or more of soft tissue or bony tissue.

3. The method of claim 1, further comprising harvesting the tumor tissue from the subject.

4. The method of claim 1, further comprising morcellating the tumor tissue harvested from the subject prior to reintroducing the tumor tissue into the subject.

5. The method of claim 4, wherein morcellating the tumor tissue comprises placing the harvested tumor tissue in a containment chamber, wherein the tumor tissue is morcellated in the containment chamber.

6. The method of claim 1, further comprising devitalizing cancer cells in the tumor tissue harvested from the subject.

7. The method of claim 6, wherein devitalizing cancer cells in the tumor tissue comprises destroying the cancer cells.

8. The method of claim 6, wherein devitalizing cancer cells in the tumor tissue comprises cooling the tumor tissue.

9. The method of claim 8, wherein the tumor tissue is cooling after being morcellated.

10. The method of claim 8, further comprising warming the cooled tumor tissue.

11. The method of claim 10, further comprising repeating one or more cool and warm cycles to the tumor tissue.

12. The method of claim 11, wherein the one or more cool and warm cycles conform to a specified range of speeds of cooling and warming, optimized to devitalize or kill cells without destroying tumor antigens.

13. The method of claim 11, wherein the tumor tissue with the cancer cells devitalized is reintroduced into the subject after said tumor tissue has reached a threshold number of cool and warm cycles.

14. The method of claim 8, wherein the tumor tissue with the cancer cells devitalized is reintroduced into the subject after said tumor tissue has reached a threshold temperature.

15. The method of claim 13, wherein the tumor tissue with the cancer cells devitalized is reintroduced into the subject after said tumor tissue has reached the threshold temperature for a predetermined period of time.

16. The method of claim 8, wherein the cooling the tumor tissue comprises cryogenically treating the tumor tissue.

17. The method of claim 16, wherein the tumor tissue is cryogenically treated with liquid nitrogen.

18. The method of claim 1, wherein the tumor tissue with the cancer cells thereof devitalized is reintroduced with a containment element.

19. The method of claim 18, wherein the containment element comprises a containment sleeve.

20. A system for treating tissue or cells for therapeutic reinfusion, the system comprising:
a cartridge for storing and processing harvested tumor tissue;
a housing including a containment chamber to removably hold the cartridge;
a cooling fluid port fluidically coupled to the containment chamber for introduction of a cooling agent into the containment chamber to cool the cartridge; and
a user interface for one or more of:
(i) displaying one or more of time, a temperature in the containment chamber, or the phase of the freezing/thawing cycle, or
(ii) controlling one or more of a timer or the temperature in the containment chamber, or
(iii) monitoring and driving characteristics of one or more freeze/thaw cycles.

21. The system of claim 20, further comprising a tissue morcellator within the cartridge, wherein the housing includes a driver for the tissue morcellator.

22. The system of claim 21, wherein the user interface is configured to display a morcellation status of the harvested tumor tissue.

23. The system of claim 21, wherein the user interface is configured to control the driver for the tissue morcellator.

24. The system of claim 20, further comprising a warm air port fluidically coupled to the containment chamber for introduction of a warm air to the containment chamber to warm or thaw the cartridge.

25. The system of claim 20, wherein the cartridge comprises a tube.

26. The system of claim 20, wherein the cartridge comprises a tray.

27. The system of claim 20, wherein the cartridge comprises a mesh cylinder.
